(19) United States
(12) Patent Application Publication
(10) Pub. No.: US 2006/0183655 A1
(43) Pub. Date: Aug. 17, 2006

(54) Surface active composition containing alcoholethoxy sulfate for use in laundry detergents and process for making it

(76) Inventors: Thorsten Bastigkeit, Scottsdale, AZ (US); Joan Bergstrom, Phoenix, AZ (US); Aleida M. Lester, Mesa, AZ (US); Pamela C. Lam, Scottsdale, AZ (US); Daniel Wood, Phoenix, AZ (US)

Correspondence Address: SNELL & WILMER, LLP, ONE ARIZONA CENTER, 400 E. VAN BUREN, PHOENIZ, AZ, 85004-2202, US

(21) Appl. No.: 11/355,206

(22) Filed: Feb. 14, 2006

Related U.S. Application Data
(60) Provisional application No. 60/653,041, filed on Feb. 14, 2005.
(60) Provisional application No. 60/725,268, filed on Oct. 11, 2005.

(51) Int. Cl. C11D 1/83; C07C 303/06

(52) U.S. Cl. 510/351; 558/31

(57) ABSTRACT

The development and method for the production of an alkyl ethoxysulfate and alkyl ethoxysulfate/ethoxylated alcohol binary surfactant system using a sulfation process. A process for producing an alkyl ethoxysulfate/ethoxylated alcohol binary surfactant system additionally comprises the step of combining the resultant alkyl ethoxysulfate with the ethoxylated alcohol feed stream.

### CROSS-REFERENCE TO RELATED APPLICATIONS

This application claims priority from U.S. Provisional Patent Application Ser. No. 60/653,041 entitled “Liquid Laundry Detergent and Process for Producing a Binary Active Surfactant for Use Therein,” filed Feb. 14, 2005 and Provisional Patent Application Ser. No. 60/725,268 entitled “Alcohol Ether Sulfate Surfactant for Use in Liquid/Powder Detergent,” filed Oct. 10, 2005 which are hereby incorporated by reference in their entirety.

### FIELD OF INVENTION

The present invention relates generally to the development of a surface active composition for use in a laundry detergent. The new material comprises an alcoholethoxy sulfate of the formula R—O—(CH2CH20)X—SO3M, wherein R is an alkyl group with a chain length of from 12 to 18 carbon atoms and contains an average number of oxyethylene groups from 5 to 9. More specifically, the present invention relates to a system and method for producing an alkyl ethoxy sulfate and an alkyl ethoxysulfate/ethoxylated alcohol binary surfactant system using a sulfation and neutralization process.

### BACKGROUND OF THE INVENTION

The manufacture and use of synthetic laundry detergents containing anionic surfactants have been documented in the patent literature. By providing good detergency, foamability and the ability to build high viscosity formulas using alcoholethoxy sulfates are finding increasing use in laundry products. However, drawbacks to the use of traditional alcoholethoxy sulfates (C12-14 and 2 moles of ethylene oxide) are: The concentrated alcoholethoxy sulfates have a very high viscosity. Therefore they are usually handled in concentrations less than 70%, and quite often in concentrations less than 30%, or in some case, through the use of a cosolvent (e.g. alcohol) which when used result in the need to handle high flashpoint material.

Alcoholethoxy sulfates also tend to be fairly yellow in color, often resulting from impurities during the sulfation process. Liquid detergents are mainly blue. If alcoholethoxy sulfates are used in high concentrations the liquid detergent tends to take on a green appearance. Therefore when blue detergents are desired, traditional alcohol ethoxylates can only be used in limited concentrations, unless further purification steps are taken, which steps can be costly and time consuming.

Traditional Alcoholethoxy sulfates tend to be adversely affected by relatively small changes in temperatures (i.e. temperatures above 150° F. tend to cause hydrolysis). There is a need for a material that is resistant to elevated temperatures for a significantly long period of time, which can result in a significant improvement in the handling and production of laundry detergents.

Traditional Alcoholethoxy sulfates also have a limited ability to avoid redeposition of clay and fat soils, which can tend to cause graying of laundry fabrics.

Thus, what is needed is a surfactant system with improved properties and a process capable of producing it. Properties such as improved whitening capability and improved color purity would allow the material to be used at higher concentrations and thus require less diluent so as to reduce shipping and packing costs without compromising the effectiveness of the detergent. Surprisingly, it was found that an alcoholethoxy sulfate with a carbon chain of from C12-18 and 5-9 moles of ethoxylene oxide.

### SUMMARY OF THE INVENTION

While the way that the present invention overcomes the disadvantages of the known art will be discussed in greater detail below, in general, the present invention provides a method of producing an improved alkyl ethoxysulfate and an improved anionic/nonionic binary surfactant system for use in laundry detergents by sulfating an ethoxylated alcohol.

It was found that an alcoholethoxy sulfate with a C12-18 chain length and 5-9 moles of EO can be handled in higher concentrations than the more traditional alcoholethoxy sulfates (C12-15 with 2 moles of ethoxylene oxide). Concentrations of the new material in upwards of 80% were found to be as flowable as the traditional material at about 70% active. The new alcoholethoxy sulfate was also found to be much less yellow when sulfated under the same conditions and gives a better whiteness maintenance when compared to the traditional sulfate. Additionally, an unexpected benefit was found during routine stability evaluations of the material. The traditional alcoholethoxy sulfate (C12-15 with 2 moles of ethoxylene oxide) tends to hydrolyze at relatively low temperatures and times (i.e. temperatures above 150° F. for 6-8 hours). The new material, alcoholethoxy sulfate (C14-15 with 7 moles of ethoxylene oxide) was found to be stable over a much greater time and temperature period (stored at 180° for 72 hours). The new product showed little degredation vs. the traditional material.

That being said, in accordance with an exemplary embodiment of the present invention, methods and systems for producing an improved alcoholethoxy sulfate (AES) are provided.

In accordance with an exemplary embodiment of the invention, wherein ethoxylated alcohol (EA) having an alkyl chain length of about 12 to about 18 and about 5 to about 9 moles of ethylene oxide are combined with SO₃ and air and reacted in a sulfating stage to form a reaction mixture containing an unstable alkyl ethoxy acid intermediate. The reaction mixture is transported to a separator stage where the unstable alkyl ethoxy acid intermediate preferably is separated from any unwanted byproducts. The alkyl ethoxy acid intermediate is thereafter transported to a neutralization stage where it is neutralized to form AES.

In accordance with another exemplary embodiment of the present invention, EA is combined with the resultant AES to form an EA/AES binary surfactant system.

In accordance with an exemplary embodiment, the present invention may comprise a system having a sulfur trioxide production stage, a sulfation stage, a separator stage, a neutralizer stage, and a byproduct management stage.

In accordance with an exemplary embodiment, the present invention may be conducted as a batch process or as a continuous process. Attached are drawing that detail the process described below.

### BRIEF DESCRIPTION OF THE DRAWINGS

Preferred exemplary embodiments of the present invention are described in conjunction with the appended drawing figures in which like numerals denote like elements, and:

FIG. 1 illustrates a flow diagram of a system for producing AES in accordance with an exemplary embodiment of the present;

FIG. 2 illustrates a flow diagram of an SO₃ formation stage in accordance with an exemplary embodiment of the present invention;

FIG. 3 illustrates a flow diagram of an exemplary embodiment of the present invention conducted as a continuous reaction process; and

FIG. 4 illustrates a flow diagram of a method for producing a binary surfactant system in accordance with an exemplary embodiment of the present invention.

FIG. 5 is a photograph illustrating the results of a hydrolysis study.

FIG. 6 is a graphical display of data from a viscosity study with respect to one composition in accordance with one embodiment of the present invention.

FIG. 7 is a further graphical display of further data from a further viscosity study with respect to one composition in accordance with one embodiment of the present invention.

### DETAILED DESCRIPTION

The description that follows is not intended to limit the scope, applicability or configuration of the invention in any way; rather, it is intended to provide a convenient illustration for implementing various embodiments of the invention. For example, although certain preferred aspects of the invention, such as techniques and apparatus for conditioning process streams, for example, are described herein in terms of exemplary embodiments, such aspects of the invention may be achieved through any number of suitable means now known or hereafter devised. Accordingly, these and other changes or modifications are intended to be included within the scope of the present invention. Thus, the detailed description herein is presented is for the purpose of illustration only.

As such, a method and system for producing alcoholethoxy sulfate (AES) for use as an anionic surfactant in a detergent composition is provided. It should be appreciated that while the present invention will be described in connection with a detergent composition, other household or personal cleaning compositions may also benefit from inclusion of the class of alcoholethoxy sulfate disclosed in the various embodiments of the present invention. Furthermore, it should be appreciated that a method for producing AES in accordance with various embodiments of the present invention is generally any method which sulfates and subsequently neutralizes ethoxylated alcohol (EA) to produce AES.

In accordance with one exemplary embodiment, the key chemical conversions are:
CH₃(CH₂)ₙ(OCH₂CH₂)ₓOH+SO₃/Air→CH₃(CH₂)ₙ(OCH₂CH₂)ₓOSO₃²⁻+Spent Air (1)
CH₃(CH₂)ₙ(OCH₂CH₂)ₓOSO₃²⁻+MOH→CH₃(CH₂)ₙ(OCH₂CH₂)ₓOSO₃M+OH⁻ (2)
where n represents the number of carbon atoms in the alkyl substrate, x is the number of moles of ethylene oxide (EO), and M is a cation.

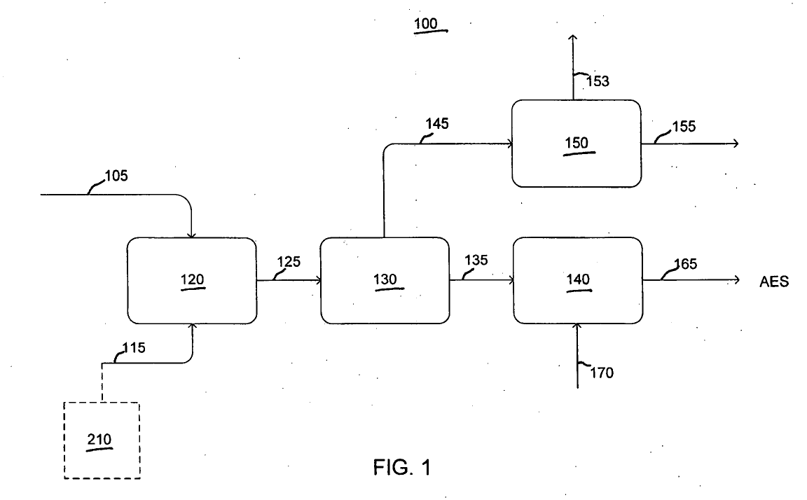

With reference now to FIG. 1, an exemplary embodiment of the present invention includes a system 100 to produce AES. In this system, preferably an EA feed stream 105 and a SO₃/air feed stream 115 are caused to react in a sulfation stage 120 to form a reaction mixture 125 containing an alkyl ethoxy acid intermediate (hereinafter an alkyl sulfuric acid). Preferably, reaction mixture 125 then is caused to flow to a separator stage 130 where the desirable alkyl sulfuric acid 135 is separated from any residual reaction components 145, such as spent air. The alkyl sulfuric acid 135 is then transported to a neutralization stage 140 where it is advantageously neutralized using a neutralizing agent 170 to form AES 165. The residual reaction components may be suitably pumped to a byproduct management stage 160 where they can be suitably treated to remove any caustic substances 153 and/or spent air 155.

In accordance with an exemplary embodiment of the present invention, EA feed stream 105 comprises an ethoxylated alcohol (EA) having a general formula of:
CH₃(CH₂)ₙ(OCH₂CH₂)ₓOH
where n is the number of carbon atoms in the alkyl substrate and x is the degree of ethoxylation, namely the number of moles of ethylene oxide (EO). It will be understood by one skilled in the art that EA typically contains a distribution of the degree of ethoxylation, and thus x represents an average degree of ethoxylation. In an exemplary embodiment, n is about 2 to about 18 and x is about 1 to about 10.

In accordance with a preferred exemplary embodiment, EA feed stream 105 comprises an ethoxylated alcohol having about 10 to about 18, preferably about 12 to about 15, and more preferably about 14 to 15 carbon atoms in the alkyl substrate and between about 4 to about 10, preferably about 6 to 8, and more preferably about 7 moles of ethylene oxide. Optimally, EA feed stream 105 comprises an ethoxylated alcohol having 14 carbon atoms in the alkyl substrate and about 7 moles of EO. Thus, a preferred EA feed stream 105 may be represented by the formula CH₃(CH₂)₁₃(CH₂CH₂O)₇OH. However, it will be appreciated that EA feed stream may comprise an ethoxylated alcohol having any number of carbon atoms in the alkyl substrate and/or moles of EO and still fall within the scope of the present invention.

Generally, the length of the alkyl substrate and the number of moles of EO will remain unchanged during the reaction process of the present invention. Therefore, in accordance with an exemplary embodiment of the present invention, the length of the alkyl substrate and the number of moles of EO in the EA are selected based upon the desired length of the alkyl substrate and moles of EO in the AES end product.

Thus, if an AES having 4 carbon atoms in the alkyl substrate and 7 moles of EO is desired, EA feed stream 105 generally will comprise an ethoxylated alcohol having 4 carbon atoms in the alkyl substrate and 7 moles of EO.

In accordance with an exemplary embodiment, the SO₃ contained in the SO₃/air feed stream 115 may be provided in any number of ways. For example, in accordance with an exemplary embodiment, the SO₃ may be purchased commercially through any known supplier.

In another exemplary embodiment, SO₃ may be prepared by heating concentrated sulfuric acid with a large excess of phosphorous pentoxide as shown by the following reaction:
H₂SO₄+P₂O₅→SO₃+2HPO₃ (3)

In accordance with another exemplary embodiment, and with momentary. reference again to FIG. 1, the SO₃ in SO₃/Air feed stream 115 optionally may be provided by an SO₃ production stage 210. SO₃ production stage 210 may comprise any apparatus, system or procedure which reacts sulfur with dry air and heat to form sulfur dioxide, SO₂, and subsequently oxidizing the sulfur dioxide to form SO₃. The key chemical conversions in SO₃ production stage 210 are:
S₂+Air→2SO₂+Spent Gas (4)
2SO₂+O₂→2SO₃ (5)

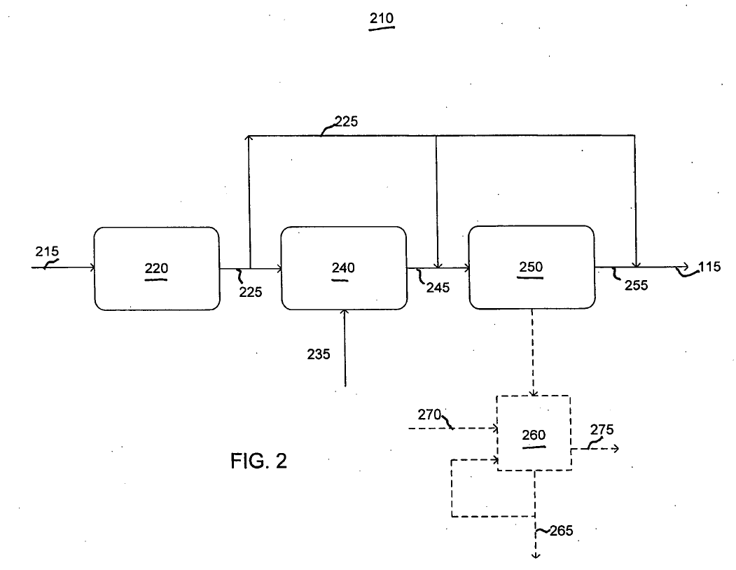

With reference now to FIG. 2, an exemplary embodiment of SO₃ production stage 210 may comprise a system where an air feed stream 215 is suitably supplied to a drying stage 220 to produce a dried air stream 225. Dried air stream 225 is then advantageously reacted with a sulfur stream 235 in a sulfur dioxide reaction stage 240 to form a resultant SO₂ stream 245. SO₂ stream 245 and dry air stream 225 are then suitably fed to an SO₃ reaction stage 250 to form SO₃ stream 255. SO₃ stream 255 may then be combined with dry air stream 225 to form SO₃/Air feed stream 115 (also shown in FIG. 1).

In accordance with an exemplary embodiment, air feed stream 215 suitably comprises ambient air and is supplied to drying stage 220 by a positive displacement blower. However, it will be appreciated that air feed stream 215 may comprise filtered or otherwise purified air, and any apparatus, system or technique capable of moving the air in air feed stream 215 into drying stage 170, such as air pumps and/or the like may be used without deviating from the scope of the invention.

Drying stage 220 may comprise any apparatus or procedure capable of purging water vapor from air, thereby preventing the formation of unwanted sulfuric acid SO₂ formation stage 240 and in SO₃ formation stage 250. For example, drying stage 220 may comprise an air dryer having an activated alumina dessicant bed. When the air passes over the dessicant bed, water vapor is transferred from the air to the dessicant bed.

In accordance with an exemplary embodiment, drying stage 220 comprises multiple air dryers so that while one dryer is drying the process air, the dessicant bed of the second dryer is being regenerated. For example, two dryers may be operated alternately on an 8-hour cycle such that a first dryer is used to dry process dry air for 4 hours while the second dryer is being regenerated. However, it will be appreciated by one skilled in the art that any time period sufficient for reconstitution of the dessicant bed may be used.

In accordance with an exemplary embodiment, the temperature of the air feed stream 215 may be decreased just prior to entering drying stage 220, for example to 60° F., using an air chiller. Preferably, the temperature in the dryer should not exceed 115° F. in order to increase air drying efficiency.

In accordance with another aspect of the present invention, air feed stream 215 may be compressed using a pressurizing means, such as a compressor, so that it is saturated with water vapor when it enters drying stage 220, in order to increase air drying efficiency.

Thus, various exemplary embodiments of drying stage 220 have been provided. However, it will be appreciated by one skilled in the art that any apparatus or procedure capable of removing water vapor from air and to produce dry air stream 225 may be used in drying stage 220.

In accordance with an exemplary embodiment, sulfur feed stream 235 comprises molten sulfur and is preferably supplied to SO₂ reaction stage 240 at a temperature of about 265° F. to about 290° F. In a preferred exemplary embodiment, sulfur feed stream 235 is stored in a steam-heated tank prior to use. However, it will be appreciated by one skilled in the art that the sulfur stored in any suitable apparatus and may be provided to SO₂ reaction stage 240 in solid, liquid and/or gaseous form.

SO₂ formation stage 240 may comprise any apparatus, system or procedure capable of atomizing sulfur and reacting it with air to form SO₂. In accordance with an exemplary embodiment, SO₂ formation stage 240 may comprise a sulfur burner of conventional construction.

SO₃ formation stage 250 may be any apparatus, system or procedure capable of oxidizing S02 to form SO₃. For example, SO₃ formation stage 250 may comprise a catalytic converter having a crushed quartzite layer and three layers of a vanadium pentoxide catalyst. The first two layers may contain, for example, Type 210 vanadium pentoxide catalyst and the third layer may contain Type LP105 vanadium pentoxide catalyst.

In accordance with an exemplary embodiment, as SO₂ stream 245 and dry air stream 225 enter the catalytic converter, they pass through the crushed quartzite layer to filter the dry air and minimize contamination of the vanadium pentoxide catalyst. SO₂ stream 245 and dry air stream 225 then pass through the three layers of vanadium pentoxide catalyst where the SO₂ is converted to sulfur trioxide (SO₃). Thus SO₃ stream 255 is formed.

As shown in the exemplary embodiment in FIG. 2, SO₃ formation stage 250 may further comprise SO₃ scrubbing stage 260.

SO₃ scrubbing stage 260 may be any apparatus,. system or procedure capable of removing SO₃ from dry air. For example, SO₃ scrubbing stage 260 may comprise an absorber. In accordance with an exemplary embodiment, during start up of SO₃ production stage 210 or shut down of sulfation stage 130, the SO₃ stream 255 may be diverted to the absorber, where it is scrubbed using water feed stream 270 to form sulfuric acid 265. Sulfuric acid 265 may be recycled through the absorber such that when the SO₃ contacts the sulfuric acid, it is absorbed and reacts with water to form alkyl sulfuric acid. In accordance with an exemplary embodiment, water may be continuously added to the scrubber to maintain a alkyl sulfuric acid concentration of 96% to 98% to maximize absorption of SO₃ and minimizes equipment corrosion. The alkyl sulfuric acid recycled through the absorber may be any concentration, but it will be understood by one skilled in the art that higher concentrations, for example 98% concentrated sulfuric acid will help to maximize absorption of SO₃.

In accordance with an exemplary embodiment, spent gas 275 exits the absorber through a demister, which removes entrained droplets of acid.

Thus, an exemplary embodiment of sulfur formation stage 250 has been provided. However, it will be appreciated that any apparatus, system or procedure capable of oxidizing SO₂ to form SO₃ may be used.

In accordance with another exemplary embodiment, spent gas 275 may be directed to byproduct management system 150 (shown in FIG. 1 and discussed below) to undergo further treatment to remove any residual caustic substances.

In accordance with an exemplary embodiment, SO₃ stream 255 may be directed to SO₃ scrubbing stage 260, sulfation stage 120 (shown in FIG. 1), or any combination of the two.

In accordance with another exemplary embodiment, SO₃ stream 255 and dry air stream 225 are then combined to form SO₃/air feed stream 115 (also shown in FIG. 1).

In an exemplary embodiment, the ratio of air to SO₃ in SO₃/air feed stream 115 is 2-5% in order to optimize the conversion of EA to alkyl ethoxy acid intermediate. However, it will be understood by one skilled in the art that ratio of air to SO₃ in SO₃/air feed stream 115 may be varied depending on the desired rate of conversion.

As shown in an exemplary embodiment in FIG. 1, EA feed stream 105 and SO₃/air feed stream 115 are suitably fed to sulfation stage 120 where they are reacted to form reaction mixture 125 which comprises a alkyl sulfuric acid and any unwanted byproduct, such as spent gases.

The key chemical reaction during sulfation stage 120 is:
CH₃(CH₂)ₙ(OCH₂CH₂)ₓOH+SO₃/Air→CH₃(CH₂)ₙ(OCH₂CH₂)ₓSO₃²⁻+Byproduct (6)
where n is the number of carbon atoms in the alkyl substrate, and x is the number of moles of EO.

In accordance with one aspect of the present invention, EA feed stream 105 and SO₃/Air feed stream 115 are transported into sulfation stage 120. In an exemplary embodiment, the mole ratio of SO₃ to EA is on the order of 1.00 to 1.04. However, it will be understood by one skilled in the art that this ratio may be determined by the necessary mole ratio of SO₃ to EA and may be adjusted depending on the feedstock of EA being sulfated and the desired yield of EA to alkyl sulfuric acid.

In an exemplary embodiment, the temperature of the SO₃/air feed stream 115 entering sulfation stage 120 may be approximately 100° F. However, it will be appreciated by one skilled in the art that SO₃/air feed stream 115 may be any temperature suitable to enable the reaction of sulfation stage 120.

Sulfation stage 120 may comprise any apparatus, system or procedure capable of reacting SO₃, air and EA to form an alkyl sulfuric acid.

In accordance with an exemplary embodiment, sulfation stage 120 comprises a Chemithon, 36-inch diameter falling film SO₃ reactor having an outer shell (barrel), an inner shell (quill), and a cooling section (bustle). A thin film of EA from EA feed stream 105 is evenly distributed on the inside of the outer shell and the outside of the inner shell of the falling film SO₃ reactor. SO₃/air feed stream 115 flows through the annular space between the outer shell and the inner shell, and reacts with the EA.

The reaction mixture then enters the cooling section of the falling film SO₃ reactor where the reaction temperature is controlled by adjusting the temperatures of SO₃/air feed stream 115 and EA feed stream 105, and the cooling jackets around the barrel and quill. In accordance with an exemplary embodiment, the cooling water in the bustle may generally be supplied at 85° F.

In accordance with an exemplary embodiment of the present invention, the temperature of the alkyl sulfuric acid stream 125 leaving sulfation stage 120 and entering separation stage 130 is on the order of about 80° F. to about 125° F. However, it will be understood by one skilled in the art that the temperature of alkyl sulfuric acid stream 125 may be varied depending on the desired yield of AES and other operating conditions.

Thus, an exemplary embodiment of SO₃ formation stage 120 has been provided. However, it will be appreciated by one skilled in the art that any apparatus, system or procedure capable of reacting SO₃, air and EA to form alkyl sulfuric acid may be used in sulfation stage 120.

In accordance with an exemplary embodiment of the present invention, any spent gases and other impurities, such as entrained alkyl sulfuric acid and sulfuric acid mist particles (hereafter “impurities”) that are generated in sulfation stage 120 may be directed to byproduct management stage 150 (discussed in detail below).

Separator stage 130 may comprise any process, apparatus or system whereby the desired alkyl sulfuric acid is separated from any unwanted impurity, such as spent gases and unreacted EA (hereafter “impurities”). In accordance with an exemplary embodiment, a cyclone may be used for this purpose. However, it will be appreciated by one skilled in the art that any number of conventional or hereafter devised separation processes and techniques may be useful to achieve the separation of the desired alkyl sulfuric acid intermediate from impurities.

After separation, the desired alkyl sulfuric acid intermediate 135 exits the separator and proceeds to neutralization stage 140 and any impurities proceed to byproduct management stage 150 (discussed below).

In accordance with an exemplary embodiment of the present invention conducted as a batch process, the desired alkyl sulfuric acid is separated from the impurities and collects in the cyclone. Once the alkyl sulfuric acid in the cyclone is filled to a pre-set level, the alkyl sulfuric acid is pumped to neutralization stage 140.

As shown in an exemplary embodiment in FIG. 1, the unstable alkyl sulfuric acid 135 is fed to neutralization stage 140 where it is reacted with neutralizer stream 170 are to form AES.

In accordance with an exemplary embodiment, the key chemical conversion in neutralization stage 140 is:
CH₃(CH₂)ₙ(OCH₂CH₂)ₓOSO ₃²⁻+MOH→CH₃(CH₂)ₙ(OCH₂CH₂)ₓOSO₃M+OH⁻ (7)
where n is the number of carbons atoms in the alkyl substrate, x is the number of moles of EO, and M is a cation.

Neutralization stage 140 may comprise any process, apparatus or system capable of reacting neutralizing stream 170 and alkyl sulfuric acid stream 145 to form AES.

In accordance with one exemplary embodiment of the present invention, neutralization stage 140 comprises a neutralizer having a mixing pump, a positive displacement recycle pump, a pH control system, and a recycle heat exchanger.

The pumps may be controlled by variable frequency drives and may be supplied by head tanks that are kept filled to minimum, specified levels. The proper pH of the mixture may be maintained by a pH control loop. The pH control loop may comprise a pH monitor with an electrode such that the pH of the neutralizer may be continuously monitored and the flow of neutralizing agent may be adjusted based on the measured pH.

In accordance with an exemplary embodiment of the present invention, neutralizer stream 170 may comprise any material capable of stabilizing the alkyl sulfuric acid. For example, neutralizer stream 170 may comprise ammonium hydroxide or sodium hydroxide. In another exemplary embodiment, neutralizer stream 195 may further comprise water, sodium bicarbonate and other additives such as propylene glycol, ammonium or sodium chloride, ammonium or sodium sulfate, ammonium or sodium bicarbonate, formaldehyde, sodium citrate, and/or tetrasodium EDTA to form AES. However, it will be appreciated by one skilled in the art that any composition capable of stabilizing the alkyl sulfuric acid may be used.

In accordance with an exemplary embodiment, the flow rates of neutralizer stream 170 and alkyl sulfuric acid stream 135 may be controlled to provide optimal conversion of the alkyl sulfuric acid. However, it will be understood by one skilled in the art that the flow rates may be determined based on the formula requirements, desired pH, and the desired rate of conversion.

In accordance with an exemplary embodiment, during start up of the process, the neutralizer may be filled with previously neutralized AES or water. The pumps for water and sodium hydroxide may be started, along with the mixing pump. The neutralizing stream 195 and alkyl sulfuric acid stream 145 may then be injected into the mixing pump, where they mix with the previously neutralized material.

The neutralized AES paste may be recycled through the heat exchanger and back to the mixing pump. A pressure control system allows neutralized paste to exit the recycle loop, so that the proper pressure can be maintained in the neutralizer. Occasionally, when higher viscosity material is produced, a booster pump, which is in parallel with the neutralizer discharge control valve, is used to maintain an acceptable pressure in the neutralizer. When neutralization is complete, the resultant AES stream 165 is transferred to a mixing tank. A sample from the tank is analyzed and, if necessary, pH adjustments are made to the AES.

In accordance with another aspect of the present invention, the resultant AES 165 may undergo further neutralization, purification and/or treatment in order to remove any residual ingredients that may have a deleterious effeci on the concentration of the AES.

As mentioned above, in accordance with an exemplary embodiment of the present invention, any residual reaction components from SO₃ production stage 210, sulfation stage 120, separation stage 130, and/or purification stage 140 may be pumped to byproduct management stage 150 to be treated to remove any impurities, especially caustic substances such as unreacted sulfur, alkyl sulfuric acid, and or sulfuric acid (hereafter “drippings”).

Byproduct management stage 150 may comprise any apparatus, system, and/or procedure capable of removing caustic substances from residual reaction components. In accordance with an exemplary embodiment, byproduct management stage 150 comprises an electrostatic precipitator (ESP). The ESP may contain, for example, a distribution plate in the bottom section to facilitate distribution of gas flow and a liquid drain. The center section may contain vertical collection tubes. An electrode mast, with seven electrode discs along its axis, may be located in the center of each collection tube. In operation, preferably, an electric corona discharge develops around the discs, and as mist particles develop a surface charge from the corona they are driven to the collection tube wall by the electrostatic field. A liquid film develops along the walls of the collection tubes and drains by gravity to the bottom of the ESP. Respective drippings 153 may be collected and deposited in the sewer.

In accordance with an exemplary embodiment, spent gas from the ESP is further purified of residual sulfur dioxide in a packed column scrubber. A dilute sodium hydroxide solution may be recirculated through the packed column scrubber to maintain a gas pressure drop. As is known, the sulfur dioxide preferably reacts with the sodium hydroxide to form sodium sulfite, which oxidizes to form sodium sulfate.

Thus, an exemplary embodiment of byproduct management stage 150 has been provided. However, it will be appreciated that any number of conventional or hereafter devised apparatus, process and/or technique suitable to treat the spent gas and other impurities may be used.

In accordance with an exemplary embodiment of the present invention, the process of the present invention may be conducted as a batch reaction process, for example when small scale production is desired, or as continuous reaction process, for example when large scale production is desired.

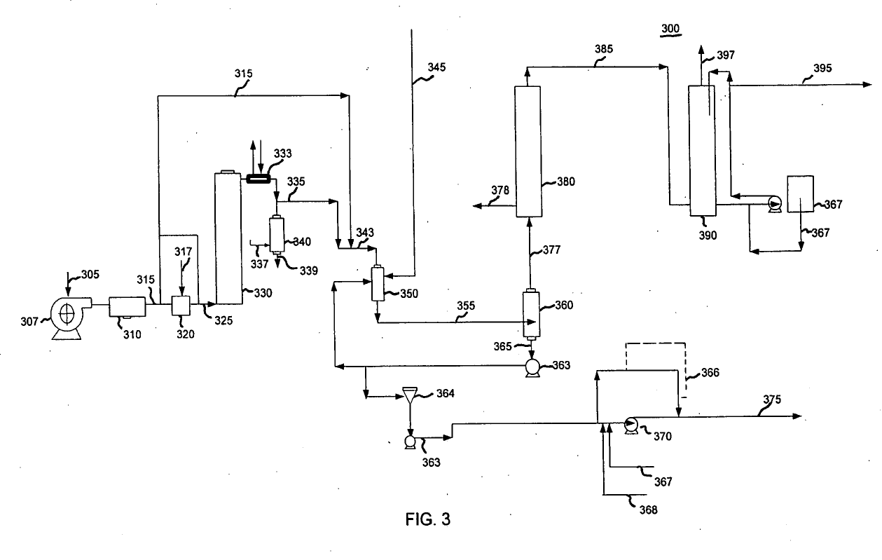

Referring to FIG. 3, an exemplary embodiment the present invention as a continuous reaction process is provided. As shown in FIG. 3, an air feed stream 305 is transported into a positive displacement blower 307 to an air dryer 310 where water vapor is removed, thereby creating the dry air feed stream 315. Dry air feed stream 315 and the sulfur feed stream 317 are then reacted, preferably in a sulfur burner 320 to produce the SO₂ stream 325. SO₂ stream 325 and dry air feed stream 315 are then reacted in a catalytic converter 330 and processed through a heat exchanger 333 to form the SO₃ stream 335. SO₃ stream 335 then is either transported to an absorber 340, where it may be reacted with sufficient amounts of water 337 to form resultant alkyl sulfuric acid 339, or it may be combined with dry air feed stream 315 to form a SO₃/air feed stream 343.

In any event, SO₃/air feed stream 343 and the EA feed stream 345 preferably are reacted in a falling film reactor 350 to form the impure alkyl sulfuric acid stream 355. Impure alkyl sulfuric acid stream 355 is then transported to a cyclone 360 where it is separated into respective alkyl sulfuric acid stream 365 and spent air stream 377.

Alkyl sulfuric acid stream 365 is either recycled back to falling film reactor 350 for further conversion or is pumped through a degasser 364 to a neutralizer 370 where it may be neutralized, such as with respective sodium bicarbonate feed stream 368 and sodium hydroxide stream 367, to form the desired AES end product 375. Optionally, pH may be monitored using a monitor 366.

A spent air stream 377 may be processed through an electrostatic precipitator 380 to remove various entrained impurities 378, and thereafter, spent air stream 385 is transported to a packed column scrubber 370 where it may be scrubbed using sodium hydroxide stream 367 to remove any additional impurities 397 to produce the substantially pure spent air stream 395.

The inventors of the present invention have found that AES made in accordance with the present invention exhibits decreased separation of components due to hydrolysis. Stated differently, AES made in accordance with the present invention retains its homogeneous dispersion of components when stored over a period of time.

### EXAMPLE 1

### Improved AES Stability

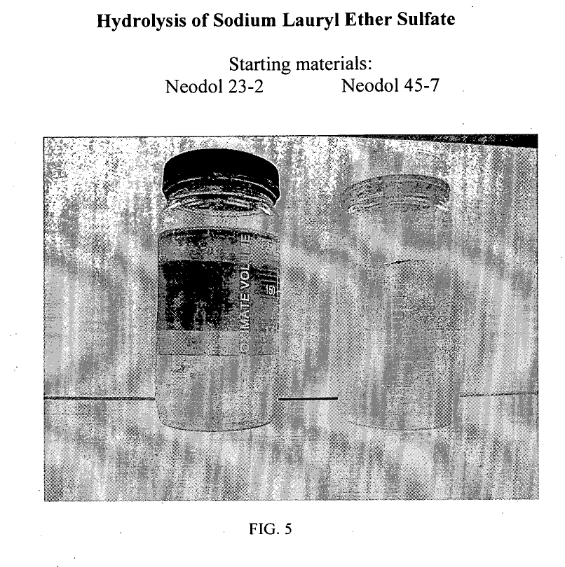

A first beaker containing approximately 4 liq. oz. of an AES produced from conventional EA and a second beaker containing approximately 4 liq. oz. of an AES produced according to the method of the present invention were stored at 90° C. for 3 days. At the end of the 3 day period, the AES in the first beaker had completely broken into its component materials of sulfuric acid and ethoxylated alcohol. The AES in the second beaker was only slightly affected by a slight drop in pH from 9.2 to 8.8 and substantially retained its homogeneous dispersion of components. These visual results are shown in the photograph comprising FIG. 5.

In accordance with another exemplary embodiment of the present invention, ethoxylate alcohol is combined with the resultant AES to form an EA/AES binary surfactant system.

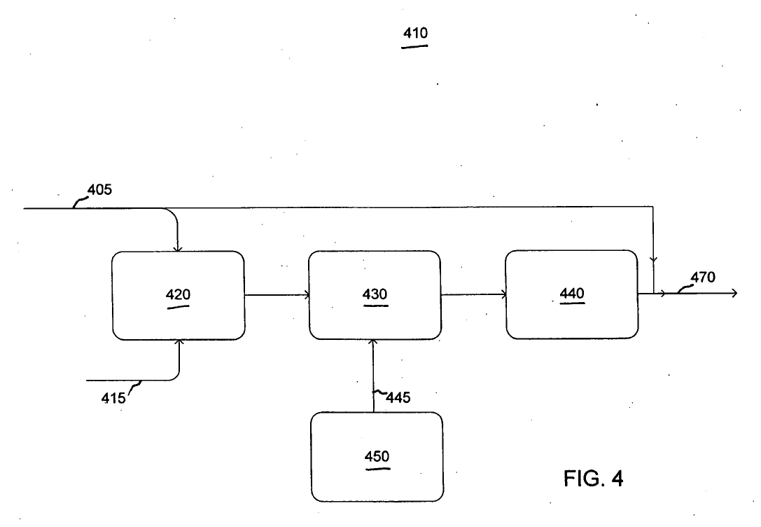

With references now to FIG. 4, an exemplary embodiment of the present invention comprises contrary EA feed stream 405 with SO₃/air feed stream 415 where it is processed through a sulfation stage 420, a separation stage 430, and a neutralization stage 440 to produce a resultant AES stream 465. According to this exemplary embodiment, EA feed stream 405 is also mixed with AES stream 465 to produce binary surfactant composition 470. Unwanted impurities 445 are processed through byproduct management stage 450.

In accordance with an exemplary embodiment, the AES and EA may be present in the binary surfactant composition 470 in a ratio of about 1:2 to about 4:1, such that the AES/EA composition ranges from about 75% of the AES to about 18% of the EA and from about 18% of the AES to about 74% of the EA. However, it will be appreciated by one skilled in the art that the ratio of AES to EA may comprise any desired ratio, depending on the desired properties, (i.e., efficacy) of the detergent.

Finally, although exemplary embodiments of the present invention are set forth herein, it should be appreciated that the invention is not so limited. Various modifications, variations, and enhancements in composition and method set forth herein may be made without departing from the spirit and scope of the present invention.

### EXAMPLE 2

### Higher Concentration

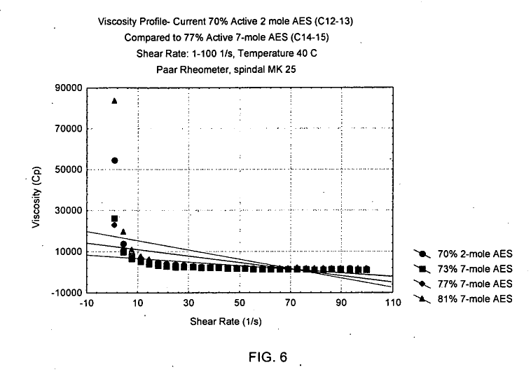

The viscosity of conventional alkyl ethoxy sulfates and the alkyl ethoxy sulfates of the present invention were also evaluated at various concentrations by varying sheer rates at a constant temperature of 40° C. as detailed in FIG. 6 attached.

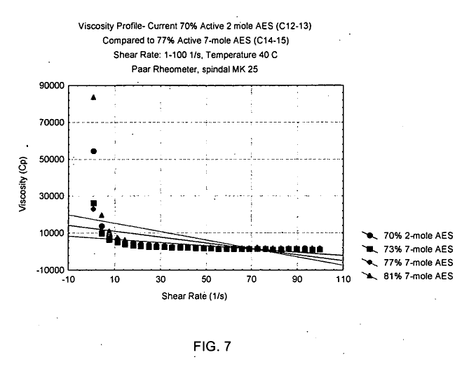

The conventional alkyl ethoxy sulfate (C12-14, EO2) at 70% concentration and the alkyl ethoxy sulfate of the present invention (C14-15, EO7) at 73-81% concentration exhibited similar viscosities although the alkyl ethoxy sulfate of the present invention was at a higher concentration as illustrated in FIGS. 6 and 7.

Various principles of the invention have been described in illustrative embodiments. However, many combinations and modifications of the above-described proportions, elements, materials and components, used in the practice of the invention, in addition to those not specifically described, may be varied and particularly adapted to specific environments and operating requirements without departing from the scope of the invention. Stated another way, the above description presents exemplary modes contemplated in carrying out the invention and the techniques described are susceptible to modifications and alternate constructions from the embodiments shown above. Other variations and modifications of the present invention will be apparent to those of ordinary skill in the art, and it is the intent of the appended claims that such variations and modifications be covered.

Consequently, it is not the intention to limit the invention to the particular embodiments disclosed. On the contrary, the invention is intended to cover all modifications and alternate constructions falling within the scope of the invention, as expressed in the following claims when read in light of the description. No element described in this specification is necessary for the practice of the invention unless expressly described herein as “essential” or “required.”

1. Surface active composition for use in a laundry detergent, which comprises
a) from about 1% by weight to about 90% by weight of a salt of an alcoholethoxy sulfate having a formula an alcoholethoxy sulfate of the formula R—O—(CH₂CH₂O)ₓ—SO₃M, wherein R is an alkyl group with an alkyl moiety from about 10 to 18 carbon atoms, M is a cation selected from the group consisting of alkali metal or ammonium ion or mixtures thereof, and x represents the average number of oxyethylene groups and is a number that varies from about 4 to about 10;
b) from 1 to about 99% water; and,
c) 0.1 to about 10% unsulfated R—O—(CH₂CH₂O)ₓ—H, inorganic and organic salts where R is selected from the group of branched or unbranched carbon groups having between about 10 and about 18 carbon atoms, and x is between about 5 to about 9.

2. The surface active composition of claim 1, wherein R is selected from said carbon containing groups having between about 12 to about 15 atoms.

3. The surface active composition of claim 2, wherein R is selected form said carbon containing groups having about 14 to 15 carbon atoms.

4. The surface active composition of claim 3, wherein x is 7.

5. The surface active composition of claim 2, wherein x is between about 6 to about 8.

6. The surface active composition of claim 3 wherein x is between about 6 to about 8.

7. A liquid detergent composition containing the surface active composition of claim 1 in a diluted form.

8. The liquid detergent composition of claim 7 wherein the surface active composition of claim 1 is utilized in diluted form.

9. A solid detergent composition containing the surface active composition of claim 1.

10. The composition of claim 1 wherein said unsulfated salt is prepared by a method comprising the steps of:
(a) providing an air and sulfur trioxide feed stream;
(b) selecting an ethoxylated alcohol having an alkyl chain length of 12-18 carbons and about 5 to about 9 moles of ethylene oxide;
(c) providing a feed stream containing said ethoxylated alcohol;
(d) reacting said air and sulfur trioxide feed stream and said ethoxylated alcohol feed stream in a thin film falling reactor to produce an alkyl sulfuric acid and byproducts;
(e) separating said alkyl sulfuric acid from said byproducts in a separator;
(f) neutralizing said alkyl sulfuric acid with neutralizer to form alkyl ethoxysulfate;
(g) combining said alkyl ethoxysulfate with said ethoxylated alcohol to form a binary surfactant system.

11. An improved binary surfactant system consisting essentially of an ethoxylated alcohol component and an alcoholethoxy sulfate component, said ethoxylated alcohol component of the formula CH₃(CH₂)ₙ(OCH₂CH₂)ₓOH, where n is a number between 2 and 18 and x is a number between about 1 to about 10, improved wherein, said ethoxylated alcohol component is produced by the method comprising the steps of:
(a) providing an air and sulfur trioxide feed stream;
(b) providing an ethoxylated alcohol feed stream;
(c) reacting said air and sulfur trioxide feed stream and said ethoxylated alcohol feed stream in a thin film falling reactor to produce alkyl sulfuric acid and spent gas;
(d) separating said alkyl sulfuric acid from said spent gas in a separator; and
(e) neutralizing said alkyl sulfuric acid with neutralizer to form alkyl ethoxysulfate
